(19) United States
(12) Patent Application Publication
(10) Pub. No.: US 2011/0005079 A1
(43) Pub. Date: Jan. 13, 2011

(54) Washable Personal Grooming Device, in Particular Hair Removal Device, and Method for the Production of Components of Such a Device

(76) Inventor: Robert Damaschke, Eschborn (DE)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/514,554

(22) PCT Filed: Sep. 29, 2007
(86) PCT No.: PCT/EP2007/008493

§ 371 Date: Dec. 21, 2009

(30) Foreign Application Priority Data
Nov. 16, 2006 (DE) 10 2006 054 027.1

(51) Int. Cl. B26B 19/38; A61K 33/38; A61P 31/04; A45D 26/00

(52) U.S. Cl. 30/41.5; 424/618; 606/133; 30/32; 30/42

(57) ABSTRACT

A washable device for personal use, in particular a hair removal device, such as an epilator and a shaver, a portion of which is coated with paraffin. The device, the paraffin, or both may include an antibacterial agent. A method of manufacturing components of a device for personal use can include dipping the components into a paraffin bath, spraying molten paraffin on the components, spraying a paraffin solution onto the components, applying solid paraffin by tumbling, and the like.

This invention relates to a washable device for personal use, in particular a hair removal device, such as an epilator and shaver, for example. The present invention relates furthermore to a method of manufacturing components of a device for personal use.

### BACKGROUND OF THE INVENTION

Devices of the type herein referred to are widely known. For example, DE 10 2004 047 874 A1 describes a hair removal device of the epilator type. Furthermore, a shaver is known from DE 41 28 217 A1. All these devices come into contact with water and other liquids repeatedly. They are cleaned or rinsed with liquid. In addition, wet shavers, for example, come into contact with liquids also during their use. The liquid penetrates through openings and minutest cavities in the devices, entering in particular inner lying regions of the housing where it remains. The liquid retained in these regions does not usually tend to dry quickly.

Such accumulation of liquid in the devices is problematic particularly when considering that in use of the devices, for example, during shaving or epilating, skin and/or hair particles are released which are entrained with the liquid and then settle in the devices. Owing to the temperatures typically prevailing in a living room or bathroom environment and the relatively long dwell time of the liquid in the devices, undesired pathogenic agents in the form of germs or bacteria may develop and propagate very rapidly. Over time, the user of the device is exposed to a considerable risk of infection.

### OBJECT OF THE INVENTION

Based on this realization it is an object of the present invention to propose a washable device for personal use with the features initially referred to, which inhibits the formation and propagation of hazardous pathogenic agents. It is a further object to propose a method of manufacturing components of such a device for personal use.

### THE INVENTION AND ADVANTAGEOUS EFFECTS

To accomplish the object, a washable device for personal use, in particular a hair removal device, is proposed, which includes the features identified in claim 1. The device is characterized, inter alia, in that it is coated with paraffin at least in part.

The technique of the invention utilizes the hydrophobic property of paraffin. This has the effect of causing the impinging liquid to bead up and roll off of the coated surfaces. Using only a slight shaking motion of the device, it is thus possible to achieve draining of the liquid as from openings and minutest cavities. The tribological properties of surfaces so treated (with paraffin, for example) are thus changed to advantage.

The dwell time of the liquid in the devices can be thereby reduced such that the development of pathogenic agents is avoided right from the outset or their propagation is at least inhibited. Health hazards to the users of the devices are thus avoided.

According to a first embodiment of the invention, surfaces of individual components of the device are coated with paraffin. With minimum effort and thus at relatively low cost the formation and propagation of pathogenic agents can be prevented by selective coating of only the surfaces of those components in which liquid accumulates and from which draining of the liquid heretofore has proven difficult.

The coated surfaces of the components suitably lie inside the housing of the device. Accordingly, those components are selected for coating in which the liquid tends to accumulate, whereby the formation and propagation of pathogenic agents is counteracted particularly effectively.

As an addition or alternative, the coated surfaces of the components may lie outside the housing of the device. This is designed to enhance the draining of the liquid also via the components lying outside the housing.

The coating of housing surfaces lying in the interior aims in the same direction.

According to another embodiment of the invention, the paraffin coating is made of hard paraffin. Hard paraffins are odorless and nontoxic, chemical resistant and exhibit a wide range of melting point settings, which makes them particularly suitable for use in washable devices for personal use.

According to another embodiment of the invention, means are provided for carrying liquid away. These means aid in draining the liquid, in particular the liquid droplets formed as a result of the paraffin coating.

Such means are suitably in the form of bores, slots, grooves and/or similar drainage devices on and/or in the device. Depending on the type of liquid accumulation, for example, in minutest cavities or within the housing of the device, a corresponding drainage device can be implemented selectively for draining the liquid effectively.

According to another embodiment of the invention, the housing and/or the components are made of plastics at least in the coated area. This enables low-cost manufacture of the device in spite of the paraffin coating. It is particularly economical to make the components completely of plastics.

According to another embodiment of the invention, the paraffin coating contains an antibacterial agent. By virtue of this approach, aside from a function conducive to draining of the liquid, the coating applied to the components and to the housing has the added effect of inhibiting the formation and propagation of pathogenic agents. This is thus an optimum way of preventing pathogenic agents, such as bacteria, germs and the like, which constitute a health hazard, from developing and propagating on the devices.

In a further aspect of the invention, the device contains an antibacterial agent. Apart from the coating, also the device or its components are thus provided with an antibacterial agent. This approach ensures that also areas devoid of coating develop an antibacterial effect so that both the formation and the propagation of pathogenic agents is likewise inhibited there. This aspect may also be considered an idea of the invention in its own right.

Preferably, the antibacterial agent is arranged at least in layers proximate to the surfaces of the device housing and/or individual components of the device in the interior and/or exterior space of the device. It is thereby ensured that the antibacterial effect develops optimally since it is precisely on or in the layers proximate to the surfaces where the pathogenic agents propagate.

Advantageously, the antibacterial agent is contained in components of the device that come into direct contact with the user's skin. This counteracts the communication of pathogenic agents to the skin which may already have developed in the device.

To be able to produce the device at low cost nevertheless, the housing and/or the components are suitably made of plastics at least in the layers proximate to the surfaces. This makes it possible in particular for the antibacterial agent to be simply molded in during manufacture of the components, thereby enabling the economical production of components including an antibacterial agent.

Advantageously, the antibacterial agent is substantially uniformly distributed to ensure a correspondingly uniform effect over the component surfaces concerned.

Advantageously, silver is provided as antibacterial agent. Silver is available, for example, on different substrates as batch for plastics as a high-volume product. As such, this antibacterial agent can be loaded in the devices at low cost. In addition, silver is said to have a particularly effective antibacterial effect. This effect is based on released silver ions which, from a certain concentration, produce an antibacterial and fungicidal effect.

Preferably, the silver content in the housing and/or in the components and/or in the paraffin coating should be greater than 5 ppm or, more preferably, greater than 20 ppm or between 10 and 130 ppm. Silver can be used to particularly good effect if the silver content is between 10 and 100 ppm. The silver content should preferably lie between 15 and 60 ppm since an optimal antibacterial and fungicidal effect is obtainable in this particular range of concentration.

According to a further aspect of the invention, on surfaces of the device which are in sliding engagement with each other, at least one of these surfaces is coated with paraffin. This makes use of the property of paraffin as lubricant. The paraffin makes it possible for the device to be lubricated for life. Wasteful relubrication is not necessary. This is substantially attributable to the advantageous properties of paraffin, making it resistant to the majority of household-type cosmetic/cleaning chemicals and resistant to mechanical cleaning as by means of brushes, in addition to being suitable for use in a wide temperature range. Paraffin is not washed out by tensides, such as soaps, dishwasher detergents, alcohols and the like, as is the case with the lubricating greases heretofore used.

Advantageously, the coated surfaces have a rough structure, preferably a structure providing a lubricant reservoir. Even in the presence of a certain amount of wear of the paraffin coating, it is still possible to ensure adequate lubrication because the surface structure has inclusions serving as paraffin reservoir.

Advantageously, the surfaces which are in sliding engagement with each other form a matching pair of metal, plastics and/or ceramic material. By means of the paraffin coating, this mating of materials enables a particularly good life lubrication to be achieved. For example, the lubrication of bearings, guides, drives and similar motion transmitting members is thereby possible without problems.

To accomplish the object, a method of manufacturing components of a device for personal use, in particular a hair removal device, such as an epilator and shaver, is also provided. This method is characterized, inter alia, by the steps of dipping the component into a paraffin bath and/or spraying molten paraffin on it, or spraying a paraffin solution onto the component, or applying solid paraffin by tumbling.

This technique enables a particularly firm and thus lasting paraffin coating to be obtained on the components for the device as, for example, a hair removal device of the epilator or shaver type.

### EMBODIMENTS

Further objects, advantages, features and application possibilities of the present invention will become apparent from the subsequent description of embodiments with reference to the accompanying drawings. It will be understood that any single feature described and/or represented by illustration, when used singularly or in any meaningful combination, forms the subject-matter of the present invention, irrespective of its summary in the claims or their back-references. In the drawings,

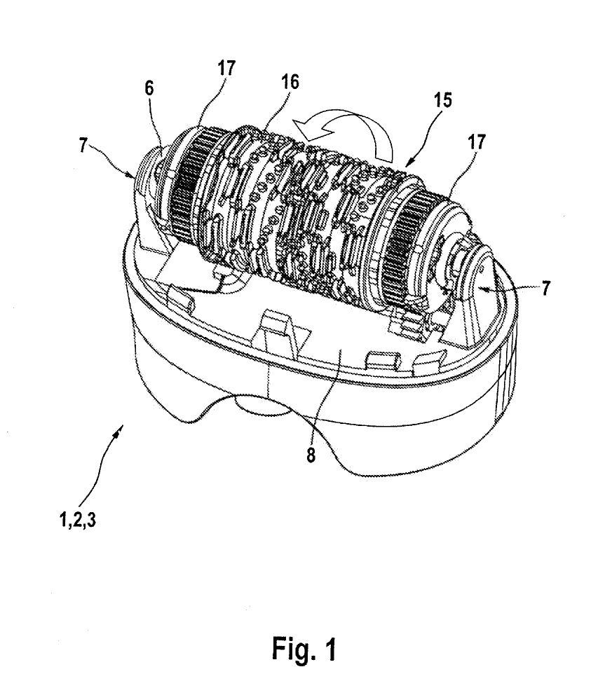

FIG. 1 is a perspective view of the upper part of a washable device of the epilator type illustrating a possible embodiment thereof;

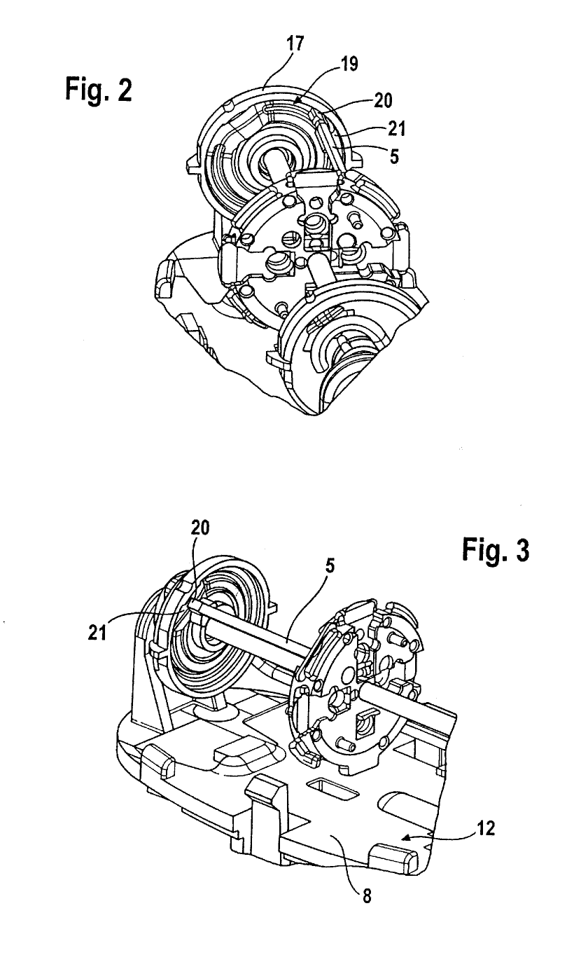

FIG. 2 is a perspective view of part of the bank of rolls of the device of FIG. 1 illustrating a first operating position;

FIG. 3 is a perspective view of part of the bank of rolls of FIG. 2 illustrating a second operating position;

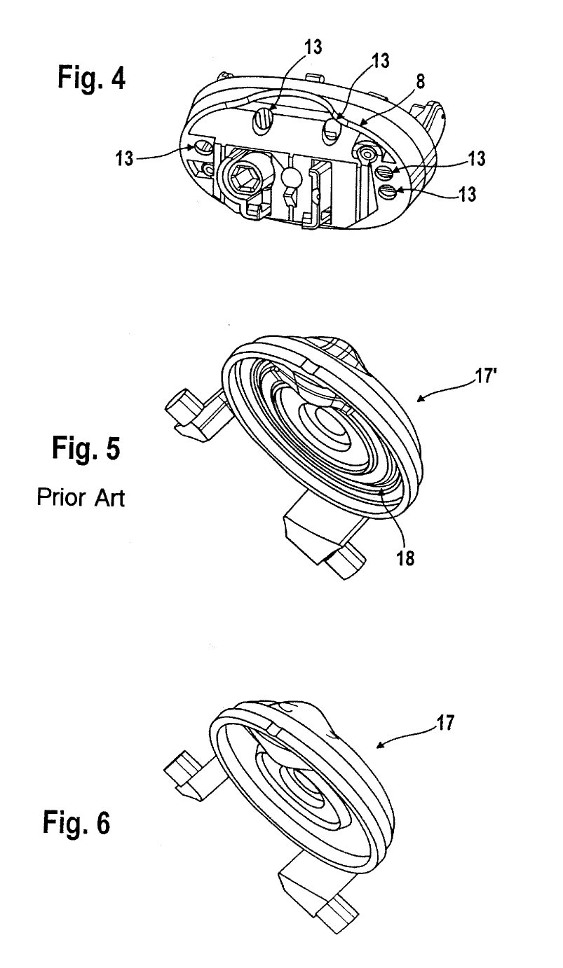

FIG. 4 is a perspective view from below of the upper part of the device of FIG. 1;

FIG. 5 is a perspective view of an end plate for an upper part of a known epilator with circumferential grooves;

FIG. 6 is a perspective view of an end plate of the epilator of FIG. 1; and

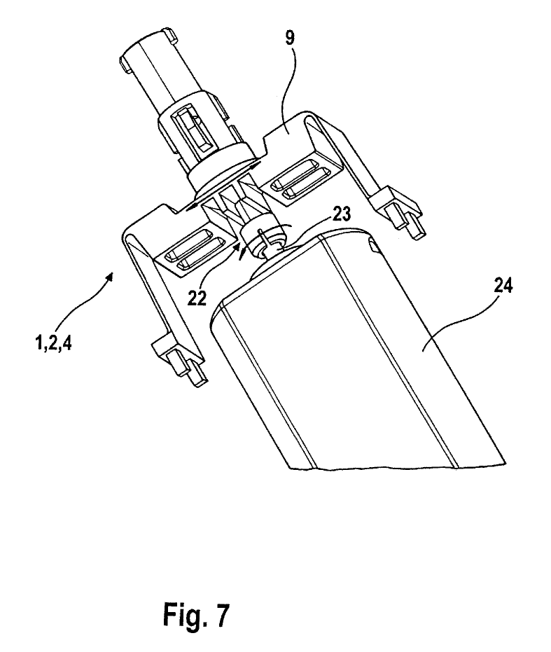

FIG. 7 is a partial perspective view of a hair removal device of the shaver type illustrating a further possible embodiment thereof.

FIGS. 1 to 3 show part of a washable device 1 for personal use of the type of a hair removal device 2. The hair removal device 2 shown in these Figures is an epilator 3 of which only its upper part or head portion is shown.

The head portion of the epilator 3 includes a bank of rolls 15 with an epilating cylinder 16 and pushrod-type components 5 located therein. The epilating cylinder 16 is held between two end plates 17, said end plates 17 transmitting an actuating force to the pushrods 5. Rotation of the epilating cylinder 16 causes the pushrods 5 to slide with one end along a predetermined curved path in a respective guide arranged on the inside of the end plates 17. Thus far, the described features are known in the art.

FIG. 2 shows, by way of example, one of the pushrods 5 in a first position abutting one of the end plates 17, and FIG. 3 shows this pushrod 5 in a second position in which the pushrod 5 has already performed a certain movement along the curved path.

In contrast to prior-art epilators, the epilator according to FIGS. 1 to 3 is coated with paraffin, preferably hard paraffin, on at least part of the epilator 3. Individual components have their surfaces coated with paraffin.

In the embodiment of FIGS. 1 to 3, the surfaces of components 7 and 8 are coated with paraffin. Components 7 are the end bearings for the epilating cylinder 16 in which liquid accumulates in the cavities particularly when rinsing the epilator 3. Component 8 is the bottom part of the head portion of the epilator 3. In combination with a cover, not shown in FIGS. 1 to 3, for the epilating cylinder 16, this component 8 forms a housing surrounding the head portion of the epilator 3.

This housing (not shown completely) may have its surface facing the epilating cylinder 16 coated with paraffin.

By coating the components of the epilator 3 as performed, for example, in the embodiment of FIGS. 1 to 3 on the components 7 and 8, the liquid impinging on these surfaces beads up and rolls off. This aids in the draining of such liquid beads from the epilator 3.

Furthermore, appropriate draining means 13 are provided as becomes apparent from FIG. 4. The means 13 for draining liquid are provided in the form of bores or openings in the bottom 8 of the head portion of the epilator 3.

In the embodiment of FIGS. 1 to 3, a paraffin coating is also provided on surfaces which are in sliding engagement with each other. The surfaces involved are, for example, the surfaces 21 of the guide 19 of the end plates 17 which are in sliding contact with the surfaces 20 of the ends of the pushrods 5. In addition to the ends of the pushrods 5, the guide 19 of the end plates 17 or the entire end plates 17 may also be coated with paraffin. The sliding surface between the ends of the shaft 6 of the epilating cylinder 16 and the end bearings 7 is also coated with paraffin. Insofar, lubrication is guaranteed by the coating. Similar to the coated components 5 and 6 and, where applicable 17, according to the embodiment of FIGS. 1 to 3, preferably such components are coated as lie within the housing (not shown) of the epilator 3.

Preferably, on the surfaces 20, 21 of pushrod 5 and guide 19, which are in sliding relationship to each other, as well as on any other relatively sliding surfaces of the epilator 3 the coated surfaces exhibit a rough structure, preferably a structure providing a lubricant reservoir. In the embodiment of FIGS. 1 to 3, the surface of the relatively sliding parts has a preferred surface roughness in a range of between 10 and 30 μm.

In the embodiment of FIGS. 1 to 3, all the paraffin coatings are loaded with a silver content of 20 to 50 ppm. The paraffin coating thus affords the possibility that liquid impinging on the surfaces beads up in droplets which then roll off, which aids in the draining of the liquid from the epilator 3. In addition, the silver content added to the paraffin produces an antibacterial effect so that the paraffin coating combined with the silver content inhibits the development of hazardous germs and their propagation.

It will be understood, of course, that the material of the components itself may contain an antibacterial agent, such as silver. The content of this agent in the material is preferably equally between 20 and 50 ppm.

The coated components 5, 6, 7, 8 are predominantly manufactured from plastics. By virtue of the high reduction in friction caused by the paraffin coating on the components 6, 7 and, respectively, 5, 17 which are in sliding relationship to each other, it is however also possible for the material of the relatively sliding surfaces to be made of plastics.

As becomes apparent from FIGS. 5 and 6, the paraffin coating enables the end plates 17 to be constructed in a simpler way. FIG. 5 shows an example of an end plate 17′ of the prior art. This end plate includes circumferential grooves 18 which are used as lubricant reservoirs for the lubricating greases used in the known end plates 17′. Such circumferential grooves are no longer necessary on the end plates 17 of the epilator 3 of FIGS. 1 to 3.

FIG. 7 shows another embodiment of the washable device 1 for personal use of the type of the hair removal device 2. The hair removal device 2 shown in this Figure is an electric shaver 4 having an oscillatory bridge 9 which includes an eccentric device 22.

This eccentric device is set in a translational motion by a rotary output shaft 23 of an electric motor 24.

In the embodiment of FIG. 7, the relatively sliding surfaces of the eccentric device 22 are coated with paraffin to thereby obtain a for-life lubrication.

Moreover, the oscillatory bridge 9 as one of the components is preferably coated with paraffin completely in order for the liquid impinging on the oscillator bridge to bead up and roll off.

It will be appreciated, of course, that also the relatively sliding surfaces of a long-hair trimmer (not shown), which is attachable to the shaver 4, may be coated with paraffin. In like manner, all the components of the long-hair trimmer as such may include a paraffin coating.

1-15. (canceled)

16. A hair removal device comprising:
a housing; and
components arranged in the housing,
wherein at least a portion of the hair removal device is coated with paraffin.

17. The hair removal device of claim 16, wherein the components are arranged to move relative to the housing, and a surface of one of the components is coated with paraffin.

18. The hair removal device of claim 16, wherein the components comprise components of an epilator, and a surface of one of the components is coated with paraffin.

19. The hair removal device of claim 18, wherein the epilator components are selected from the group consisting of pushrods located in an epilating cylinder, a shaft of an epilating cylinder, end bearings for an epilating cylinder, and a head portion of the epilator.

20. The hair removal device of claim 16, wherein the components comprise components of an electric shaver, and a surface of one of the components is coated with paraffin.

21. The hair removal device of claim 20, wherein the electric shaver components comprise an oscillating bridge.

22. The hair removal device of claim 16, wherein the coated portion of the hair removal device comprises an interior surface of the housing.

23. The hair removal device of claim 16, wherein the paraffin comprises hard paraffin.

24. The hair removal device of claim 16, wherein the device is configured to allow liquid to drain from the device.

25. The hair removal device of claim 24, wherein a surface of the device defines a recess configured to allow liquid to drain from the device.

26. The hair removal device of claim 24, wherein a surface of the device defines an opening configured to allow liquid to drain from the device.

27. The hair removal device of claim 16, wherein the coated portion of the device comprises plastic.

28. The hair removal device of claim 16, wherein the paraffin comprises an antibacterial agent.

29. The hair removal device of claim 28, wherein the device is configured such that the paraffin comprising the antibacterial agent is arranged to come into direct contact with a user's skin.

30. The hair removal device of claim 29, wherein the housing comprises plastic proximate a surface of the housing.

31. The hair removal device of claim 28, wherein the antibacterial agent comprises silver.

32. The hair removal device of claim 31, wherein the silver content in the paraffin is greater than 5 ppm.

33. The hair removal device of claim 16, wherein two of the components define surfaces that are in sliding engagement with each other, and one of said surfaces is coated with paraffin.

34. The device of claim 33, wherein the surface that is coated with paraffin defines a multiplicity of lubricant reservoirs before coating.

35. The hair removal device of claim 33, wherein the surfaces in sliding engagement with each other are fabricated from substantially the same material.
